(19) United States
(12) Patent Application Publication
(10) Pub. No.: US 2010/0113998 A1
(43) Pub. Date: May 6, 2010

(54) PELVIS SUPPORTER

(75) Inventor: Hirohisa Mizumoto, Osaka (JP)

Correspondence Address: TROUTMAN SANDERS LLP;5200 BANK OF AMERICA PLAZA, 600 PEACHTREE STREET, N.E., SUITE 5200, ATLANTA, GA, 30308-2216, US

(73) Assignee: MIZUNO CORPORATION, Osaka (JP)

(21) Appl. No.: 12/610,722

(22) Filed: Nov. 2, 2009

(30) Foreign Application Priority Data
Nov. 4, 2008 (JP) 2008-283412
Jun. 15, 2009 (JP) 2009-142144

(51) Int. Cl. A61F 5/00

(52) U.S. Cl. 602/23

(57) ABSTRACT

A pelvis supporter is wearable for exercise, and includes a base garment to be worn on a lower body of a wearer and a resin pattern formed tightly adhered on a surface of the base garment and extending annually around wearer's hip when worn. The resin pattern may have a high density area of high pattern density and a low density area of low pattern density, and it may have a wide area of wide pattern width and a narrow area of narrow pattern width.

This nonprovisional application is based on Japanese Patent Application No. 2008-283412 filed on Nov. 4, 2008 and No. 2009-142144 filed on Jun. 15, 2009, with the Japan Patent Office, the entire contents of which are hereby incorporated by reference.

### BACKGROUND OF THE INVENTION

1. Field of the Invention

The present invention relates to a pelvis supporter wearable when practicing exercise, enabling securing/orthopedic correction of pelvis.

2. Description of the Background Art

Conventionally, pelvis correction belt has been known, which is wrapped around one's hip and pelvic parts to be used for orthopedically correcting pelvic distortion or strain. When one uses such a pelvis correction belt, he/she can tighten up iliac bone and sacroiliac joint at the pelvic part from outside, so as to correct distortion or strain of pelvis and to alleviate disorders of his/her body such as low-back pain.

An example of such a pelvis correction belt is described in Japanese Patent Laying-Open No. 2001-87295 (Patent Document 1). The pelvis correction belt described in the document includes an upper belt mainly covering iliac crests and sacroiliac joint, and a lower belt mainly covering hip joint. Because of such a structure, it is possible to wrap the lower belt around left and right sides of one's body and the lower side of one's buttocks, so that the problem of displacement of pelvis correction belt moving upward from the place can be solved.

The pelvis correction belt disclosed in Patent Document 1, however, is intended to be worn for sedentary work, bedtime or general day-to-day operations, and active exercise wearing the pelvis correction belt is not expected. Therefore, when one actually practices exercise in the water or on the ground wearing the pelvis correction belt described in Patent Document 1, the pelvis correction belt will not stay in place but is displaced, moving upward from the original wearing position.

### SUMMARY OF THE INVENTION

The present invention was made in view of the foregoing and its object is to provide a pelvis supporter capable of keeping stable state of wearing even when the wearer practices exercise.

The pelvis supporter in accordance with the present invention is a pelvis supporter wearable for exercise. The pelvis supporter includes a base garment to be worn on a lower body of a wearer, and a resin pattern formed tightly adhered on a surface of the base garment and extending annually around wearer's hip when worn.

Preferably, the resin pattern has a high density area of high pattern density and a low density area of low pattern density. By way of example, the high density area may be arranged on a central front portion, a central rear portion and side portions of the base garment, and the low density area may be arranged between the central front portion and the side portions and between the central rear portion and the side portions.

Preferably, the resin pattern has a wide area wide in a direction orthogonal to the direction of extension of the resin pattern and a narrow area narrow in the orthogonal direction. By way of example, the wide area may be arranged on the central front portion and the central rear portion, and the narrow area may be arranged between the central front portion and the central rear portion.

Since the pelvis supporter in accordance with the present invention includes the annular resin pattern formed on the surface of base garment, appropriate fastening force can be applied by the resin pattern to the pelvis of the wearer when one wears the pelvis supporter. Further, since the resin pattern is formed tightly adhered on the surface of base garment, stable state of wearing can be kept even at the time of exercise.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a front view of the pelvis supporter in accordance with an embodiment of the present invention.

FIG. 2 is a side view of the pelvis supporter in accordance with the embodiment of the present invention.

FIG. 3 is a rear view of the pelvis supporter in accordance with the embodiment of the present invention.

FIG. 4 is an enlarged view of an area 11 of FIG. 1.

FIG. 5 is a cross-sectional view showing an exemplary structure of a linear pattern forming the resin pattern.

FIG. 6 is a flowchart representing a method of manufacturing the pelvis supporter in accordance with the embodiment of the present invention.

FIG. 7 is a front view of the pelvis supporter in accordance with another embodiment of the present invention.

FIG. 8 is a rear view of the pelvis supporter in accordance with another embodiment of the present invention.

### DESCRIPTION OF THE PREFERRED EMBODIMENTS

In the following, embodiments of the present invention will be described with reference to the figures. In each of the embodiments, elements attaining the same function are denoted by the same reference characters and description thereof will not be repeated unless necessary.

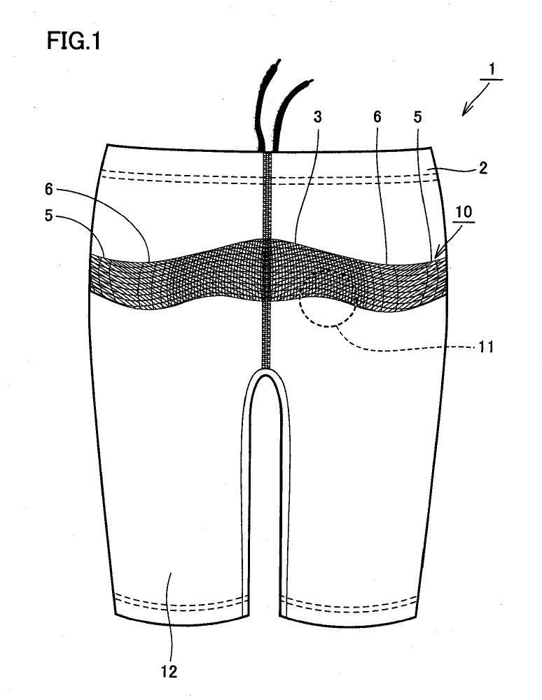

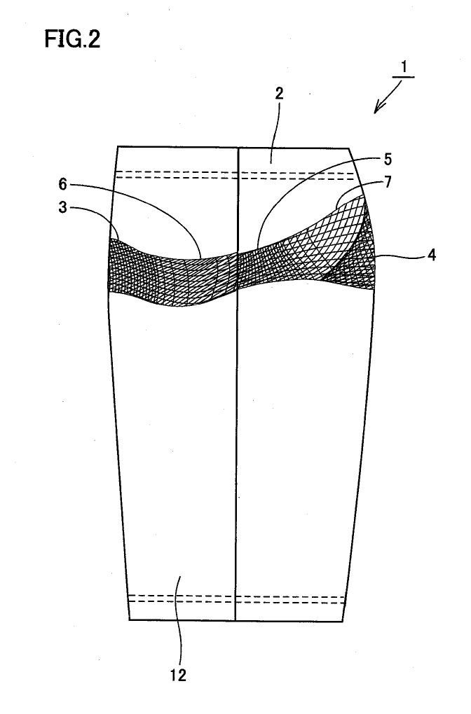

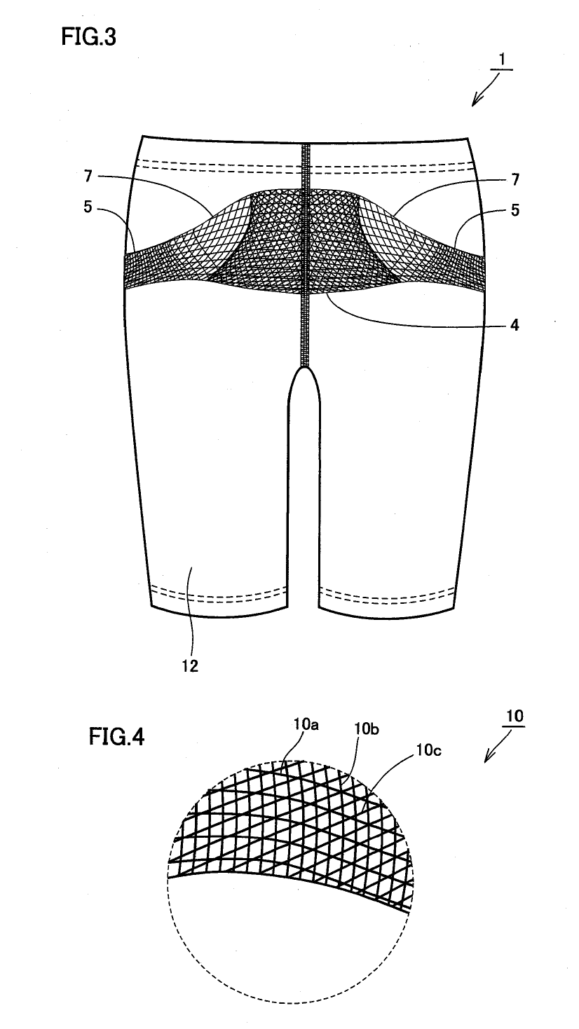

FIG. 1 is a front view of a pelvis supporter 1 in accordance with an embodiment of the present invention. FIG. 2 is a side view of pelvis supporter 1 in accordance with the embodiment of the present invention. FIG. 3 is a rear view of pelvis supporter 1 in accordance with the embodiment of the present invention. FIG. 4 is an enlarged view of an area 11 of FIG. 1.

Pelvis supporter 1 in accordance with the present embodiment is usable not only for light exercise such as walking in the water or on the ground, but also for heavy exercise including aquatic sports and track and field events.

As shown in FIGS. 1 to 3, pelvis supporter 1 includes a base garment 2 wearable around the lower parts of the wearer, and a resin pattern 10 formed tightly adhered on a surface of base garment 2, extending annually around the hip of the wearer when worn.

Base garment 2 has a body portion that can receive crotch, buttocks and part of lower limbs of the wearer, a waist portion positioned on one end side of the body portion and comes close to the lower abdomen of the wearer when worn, and hem portions 12 positioned on the other end side of the body portion and come on the lower limbs of the wearer when worn.

Base garment 2 is preferably formed of a stretch fabric to facilitate various movements. Base garment 2 may be formed by one type of stretch fabric, or it may be formed by combining a number of different types of stretch fabrics. Alternatively, a stretch fabric may be used as a base material and a non-stretch fabric may be partially used. As the clothing fabric for base garment 2, knitted fabric or woven fabric combining fibers selected, for example, from polyurethane fiber, polyamide fiber and polyester fiber may be used.

In the example shown in FIG. 1, base garment 2 has a shape of shorts, with hems 12 of body portion almost reaching knees of the wearer. The base garment 2, however, may have any shape other than the shorts shape. By way of example, it may have a structure of briefs, with hems 12 covering only the portions near hip joints when worn, or it may have a structure of leggings that almost entirely covers lower limbs with hems 12 reaching near Achilles tendons or heels. As described above, length of portions of body portion of base garment 2 covering the lower limbs of the wearer may be changed arbitrarily, and the shapes of body portion, waist portion and hems 12 of base garment 2 may be selected arbitrarily. Further, it is preferred to attach a drawstring or a rubber band at the waist portion so that pelvis supporter 1 can be secured on the waist portion of the wearer when worn.

Since pelvis supporter 1 has base garment 2 as described above, when the wearer practices ordinary exercise or even when he/she moves his/her body and limbs hard, significant displacement of pelvis supporter 1 upward or downward from the original wearing position can be prevented. Therefore, even during exercise, it does not move significantly, and stable state of wearing can be maintained.

When hems 12 are adapted to be in close contact with the lower limbs of wearer, possibility of significant displacement of the areas covering the lower limbs upward or downward from the original wearing position can further be reduced. Further, as to that portion of base garment 2 which is positioned upper than the crotch of the wearer when worn, significant displacement upward from the original wearing position can be prevented as the crotch of the wearer interferes with the crotch portion of base garment 2.

Resin pattern 10 may be formed of elastic resin material such as urethane resin. Further, resin materials such as rubber, silicone, PVC (polyvinyl chloride), polyamide elastomer, polyester elastomer, rubber-based elastomer, olefin-based elastomer, polyethylene, polypropylene, nylon, EVA (ethylene-vinyl acetate copolymer), or ABS (acrylnitrile-butadiene-styrene copolymer) may be used as the material for resin pattern 10.

As shown in FIGS. 1 to 3, resin pattern 10 may be formed by a number of linear patterns, or resin pattern 10 may be formed by combining linear patterns and planar patterns of arbitrary shapes. Material, shape and the like of each of the linear patterns and the like may be arbitrarily selected.

As shown in the enlarged view of FIG. 4, in the examples of FIGS. 1 to 3, resin pattern 10 is formed by a plurality of linear patterns arranged to intersect with each other on a surface of base garment 2. By way of example, in the area shown in FIG. 4, resin pattern 10 has such a structure that three types of substantially linear patterns 10a, 10b and 10c extending in different directions intersect with each other.

Resin pattern 10 of the portion shown in FIG. 4 is formed by arranging a plurality of linear patterns in a net-like fashion. By appropriately adjusting the material, numbers, width and the like of linear patterns, fastening force of resin pattern 10 may be adjusted in multiple stages. For example, at a portion that requires larger fastening force, the number of linear patterns is increased to reduce exposed area of the body portion of base garment 2, while at a portion requiring only a small fastening force, the number of linear patterns may be reduced to increase the exposed area of the body portion of base garment 2. The clothing fabric forming base garment 2 has higher flexibility than the resin forming resin pattern 10, and it allows easy deformation. Therefore, by adjusting the number of linear patterns as described above, fastening force of resin pattern can easily be adjusted.

As shown in FIGS. 1 to 3, while the linear patterns extend in various directions and intersect with each other, when viewed as a whole, resin pattern 10 extends in the lateral direction (left-right direction of FIGS. 1 to 3) along the waist portion of base garment 2, and has the annular shape as mentioned above.

In the example shown in FIGS. 1 to 3, the linear patterns forming resin pattern 10 are connected to each other and, therefore, by resin pattern 10, appropriate fastening force can be applied to the pelvis of the wearer when he/she wears pelvis supporter 1. The fastening force of resin pattern secures the pelvis and the wearer can practice exercise with his/her pelvis securely held. Further, using pelvis supporter 1 in accordance with the present embodiment, orthopedic correction of pelvis is also possible.

When one wears pelvis supporter 1, resin pattern 10 applies the fastening force to the wearer through base garment 2. Therefore, fitting of base garment 2 on the wearer's hip can be improved. This may also contribute to stabilization of wearing position of pelvis supporter 10.

As shown in FIGS. 1 to 3, it is unnecessary to have the density of resin pattern 10 uniform in entire areas. In the example of FIGS. 1 to 3, resin pattern 10 generally has a high-density area in which pattern density is high, a middle-density area in which pattern density is moderate, and a low-density area in which pattern density is low.

More specifically, on a front side (foreside) of pelvis supporter 1 shown in FIG. 1, the pattern density is the highest at a central front portion 3 of base garment 2, and the pattern density of left and right side portions 5 is the next highest, second to the pattern density of central front portion 3. The pattern density is the lowest at intermediate front areas 6 between central front portion 3 and left and right side portions 5.

On the rear side (backside) of pelvis supporter 1 shown in FIG. 3, the pattern density is the highest at a central rear portion 4 of base garment 2, and the pattern density of left and right side portions 5 is the next highest, second to the pattern density of central rear portion 4. The pattern density is the lowest at intermediate rear areas 6 between central rear portion 4 and left and right side portions 5.

Here, central front portion 3 of resin pattern 10 is an area corresponding to the central portion of one's abdomen where lower abdominal muscle group including abdominal rectus muscle exists, and central rear portion 4 is an area corresponding to the back of central front portion 3, when worn.

Since the density of resin pattern 10 is made different in this manner, the fastening force applied by resin pattern 10 can be varied in the direction where resin pattern 10 extends. Further, as the fastening force by resin pattern 10 is varied in this manner, base garment better fits the wearer's body at desired portions, whereby fitting feeling of pelvis supporter can be improved when worn. On the other hand, by appropriately adjusting densities of various portions of resin pattern 10, easier movement during exercise can be attained, allowing various operations of the wearer easily when worn.

As in the example shown in FIGS. 1 to 3, when the pattern density of resin pattern 10 is made high at central front portion 3, it is possible to apply relatively large fastening force to the central portion of one's abdomen, so that the abdominal pressure can be increased. Further, it is possible to attain better fitting feeling at the central portion of the wearer's abdomen.

Further, as the pattern density of resin pattern 10 is made high at central rear portion 4, it is possible to apply relatively large fastening force to the central portion of one's back (for example, an area close to the lumbar spine and sacral bone). Further, it is possible to attain better fitting feeling at the central portion of the wearer's back.

Further, as the pattern density on side portion 5 of base garment 2 is made higher, it is possible to apply a relatively large fastening force to the side portion of wearer's body (for example, an area close to gluteus maximus and gluteus medius muscles).

On the other hand, since pattern density is low at front intermediate areas 6 and and rear intermediate areas 7, tight-fitting feeling around the wearer's hip caused by the existence of resin pattern 10 can be suppressed and at the same time, flexible movement of hip and lower limbs is possible.

Specifically, in the example shown in FIGS. 1 to 3, relatively large fastening force is applied to front, rear, left and right four portions around the wearer's hip to improve fitting feeling, while easiness of movement of the wearer is ensured at other portions. The number and positions of high-density and low-density areas can be arbitrarily selected, and the pattern density of each area can be varied in arbitrary steps.

As shown in FIGS. 1 to 3, the width of resin pattern 10 in the direction orthogonal to the direction of extension of resin pattern 10 is not necessarily uniform in the entire area, and the width may be varied in the extending direction of resin pattern 10.

In the example shown in FIGS. 1 to 3, resin pattern 10 has a wide area wide in the direction orthogonal to the direction of extension of resin pattern 10 (that is, up-down direction of FIGS. 1 to 3) and a narrow area of narrow width.

More specifically, in the example shown in FIGS. 1 to 3, central front portion 3 of resin pattern 10 is the wide area having wide width. By such an arrangement, it follows that the central portion of abdomen will be covered by the wide area when one wears pelvis supporter 1. Therefore, fastening force can be applied to the wide area at the central portion of abdomen through the clothing fabric when one wears the supporter, and base garment 2 better fits the body. This also contributes to good fitting feeling when worn.

Further, central rear portion 4 of resin pattern 10 is also the wide area having wide width. In this area also, good fit of base garment 2 and the body can be ensured widely, and this also contributes to good fitting feeling when worn.

On the other hand, areas positioned between central front portion 3 and central rear portion 4 of resin pattern 10 (for example, areas corresponding to left and right sides of one's body when worn) are narrow areas of relatively narrow width. In the example shown in FIGS. 1 to 3, side portions 5 of resin pattern 10 are made narrow areas. Therefore, when one wears pelvis supporter 1, it is as if sides of his/her body are fastened by a narrow member such as a string or a belt and, therefore, easiness of movement can be ensured while good fitting feeling of wearing is maintained.

As shown in FIGS. 1 to 3, resin pattern 10 extends curving in the direction along the waist portion of base garment 2. More specifically, resin pattern 10 has a unique wavy (curved) shape, which once goes away from the waist portion as it extends from central front portion 3 to side portion 5 of base garment 2, comes closer to the waist portion again at side portion 5, and further closer to the waist portion as it extends from side portion 5 to central rear portion 4. As a result, central front portion 3 of resin pattern 10 is further away from the waist portion as compared with central rear portion 4. Since resin pattern 10 has such a shape, it is possible to apply appropriate fastening force around the hip, when one wears pelvis supporter 1.

As described above, by varying density, width and positions of forming resin pattern 10 formed tightly adhered on the surface of base garment 2 of pelvis supporter 1, it becomes possible to apply desired fastening force to desired portions while maintaining easiness of movement and good fitting feeling when worn. As a result, it is possible to ensure the function of a supporter and to maintain stable state of wearing even when the wearer practices exercise.

Next, referring to FIG. 5, an exemplary structure of the linear pattern forming resin pattern 10 above will be described. FIG. 5 is a cross-sectional view showing an exemplary structure of a linear pattern 10a forming resin pattern 10.

In the example shown in FIG. 5, the cross-sectional shape of linear pattern 10a is approximately rectangular. More specifically, linear pattern 10a has a side surface 8 rising substantially vertically from the surface of base garment 2, with a pointed upper corner 9. Upper corner 9 of linear pattern 10a may be rounded as indicated by a dotted line in FIG. 5.

The force necessary to stretch linear pattern 10a corresponds to the fastening force caused by linear pattern 10a. The force is in proportion to the volume of linear pattern 10a. Here, as linear pattern 10a has side surface 8 rising substantially vertically from the surface of base garment 2, sufficient volume of linear pattern 10a can be ensured while the area for forming linear pattern 10a on base garment 2 is kept small. In other words, it is possible to attain desired fastening force by linear pattern 10a while the area for forming linear pattern 10a is kept small.

Height (thickness) and width of linear pattern 10a are arbitrarily adjustable. By appropriately adjusting these, it is also possible to adjust the volume of linear pattern 10a and to adjust the fastening force of linear pattern 10a.

It may be required to differentiate fastening forces applied to various portions by resin pattern 10 considering body types and preferences of wearers. Since the fastening forces applied by resin pattern 10 to various portions can easily be varied by appropriately adjusting the height (thickness), width, density and the like of linear pattern 10a forming resin pattern 10 as described above, such requirement can readily be met.

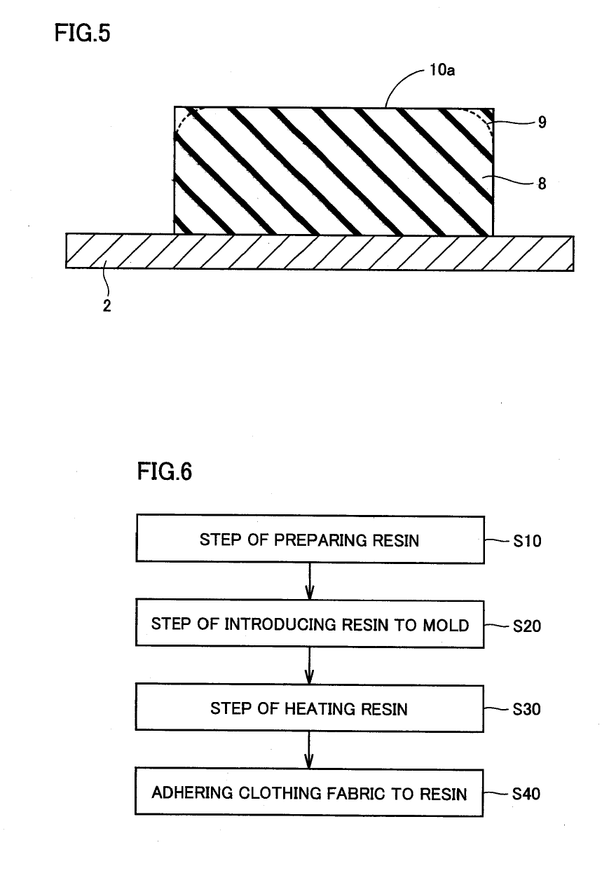

Next, the method of manufacturing pelvis supporter 1 in accordance with the present embodiment will be described with reference to FIG. 6. FIG. 6 is a flowchart representing the method of manufacturing pelvis supporter 1 in accordance with the embodiment of the present invention.

As shown in FIG. 6, first, a resin for forming resin pattern 10 is prepared (S10).

By way of example, urethane resin having prescribed elasticity and believed to have good forming characteristic is prepared.

Next, the resin is introduced to a mold (S20). The mold has a cavity corresponding to the shape of resin pattern 10, and the resin is introduced to the cavity. When the resin is introduced to the mold, the resin is set to a flowable state (for example, liquid state), to be poured to the mold. Thereafter, the resin is heated in the mold (S30). For example, it is heated at a temperature not lower than the curing temperature of the resin. Thus, part of the resin (at a portion in contact with the mold and its vicinity) is cured, or the resin as a whole is semi-cured.

Thereafter, the resin in the above-described state is brought to be tightly adhered on clothing fabric (for example, stretch fabric) for forming base garment 2 (S40). Specifically, the clothing fabric is put on the resin material that is in the heated state in the mold, and pressure is applied to the clothing fabric so that the clothing fabric comes into contact with the resin. By curing the resin in this state, a desired resin pattern can be formed on the surface of clothing fabric. It is also possible to from resin pattern 10 using a mold and thereafter to tightly adhere the resin pattern on the clothing fabric using an adhesive.

By forming resin pattern 10 on the surface of clothing fabric using a mold as described above, it is possible to form, for example, a linear pattern 10a of which side surface 8 extends in a direction substantially vertical to the surface of clothing fabric, as shown in FIG. 5.

By forming resin pattern 10 on the surface of clothing fabric in the manner as described above and thereafter by sewing up the clothing fabric, pelvis supporter such as shown in FIGS. 1 to 3 can be formed.

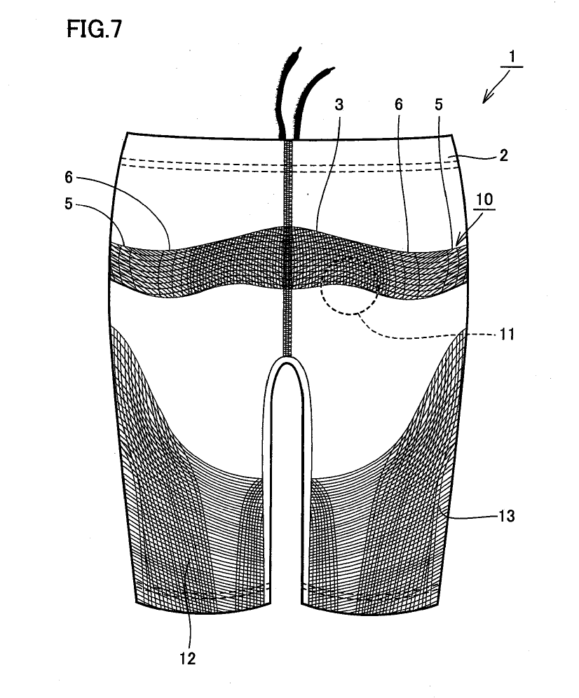

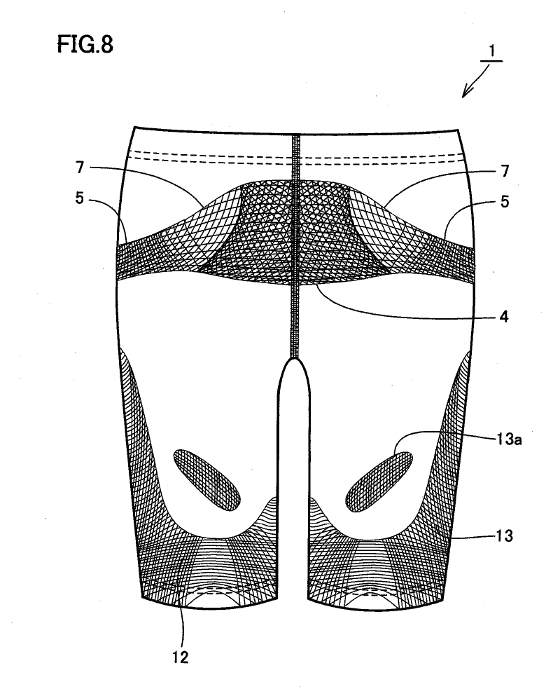

Next, another embodiment of the present invention will be described with reference to FIGS. 7 and 8.

FIG. 7 is a front view of pelvis supporter in accordance with another embodiment of the present invention and FIG. 8 is a rear view thereof.

In the present embodiment, in addition to resin pattern (first resin pattern) 10 around the hip, resin patterns 13 separate from resin pattern 10 are formed around the thighs. In the example shown in FIGS. 7 and 8, resin pattern (second resin pattern) 13 is arranged at a position covering muscle belly or/and vicinity of muscle belly of at least one muscle of a group of muscles including quadriceps muscle, hamstring, gracilis muscle, sortorius muscle and iliotibial band.

The group of muscles is attached to the pelvis and relates to the function of pelvis, in cooperation of the group of abdominal muscles and gluteus muscles. By appropriately tightening up the group of muscles by the resin pattern from outside, the pelvis can more effectively be held stable. Further, by providing resin patterns 13 around the thighs in addition to resin pattern 10 around the hip, the pelvis can be held stable while appropriate fastening force is applied around the hip.

As in the case of resin pattern 10, density of resin pattern 13 shown in FIGS. 7 and 8 may also be made different in the direction around the thigh. By way of example, density of resin pattern 13 positioned at the central portion in the widthwise direction of the thigh may be made higher than the density of resin pattern 13 positioned on the outer side or inner side in the widthwise direction of the thigh. In an opposite manner, density of resin pattern 13 positioned at the central portion in the widthwise direction of the thigh may be made lower than the density of resin pattern 13 positioned on the outer side or inner side in the widthwise direction of the thigh.

As shown in FIGS. 7 and 8, in the present embodiment, the height of resin pattern 13 positioned on the outer side in the widthwise direction of the thigh is made higher than that of resin pattern positioned on the inner side in the widthwise direction, so that resin pattern 13 extends diagonally from the outer side to the inner side portion in the widthwise direction.

Though resin pattern 13 is formed continuously in a ring-shape around the thigh upper than one's knee in the present embodiment, resin pattern 13 may be provided with a discontinuous portion such as a cut-out. Further, as shown in FIG. 8, an independent resin pattern 13a may be provided at a position covering a muscle belly or the vicinity of muscle belly of the group of muscles.

Though embodiments of the present invention have been described, it is expected from the beginning to appropriately combine structures of the embodiments above. Further, the structures of each of the embodiments may be partially omitted.

Although the present invention has been described and illustrated in detail, it is clearly understood that the same is by way of illustration and example only and is not to be taken by way of limitation, the scope of the present invention being interpreted by the terms of the appended claims.

What is claimed is:

1. A pelvis supporter wearable for exercise, comprising:
a base garment to be worn on a lower body of a wearer; and
a resin pattern formed tightly adhered on a surface of said base garment and extending annually around wearer's hip when worn.

2. The pelvis supporter according to claim 1, wherein
said resin pattern has a high density area of high pattern density and a low density area of low pattern density; and
said high density area is arranged on a central front portion, a central rear portion and side portions of said base garment, and said low density area is arranged between said central front portion and said side portions and between said central rear portion and said side portions.

3. The pelvis supporter according to claim 1, wherein
said resin pattern has wide areas wide in a direction orthogonal to the direction of extension of said resin pattern and narrow areas narrow in said orthogonal direction; and
said wide areas are arranged on said central front portion and said central rear portion, and said narrow areas are arranged between said central front portion and said central rear portion.

4. The pelvis supporter according to claim 1, wherein
said resin pattern has a wavy shape, and extends curving in a direction along a waist portion of said base garment.

5. The pelvis supporter according to claim 1, wherein
said resin pattern has a side surface rising substantially in a vertical direction from the surface of said base garment.

6. The pelvis supporter according to claim 1, wherein
said base garment covers at least a part of wearer's thigh;
said supporter further comprising
another resin pattern formed spaced apart from said resin pattern, tightly adhered on the surface of said base garment and extending around the wearer's thigh when worn.

7. The pelvis supporter according to claim 6, wherein
said another resin pattern tightens up at least one of a group of muscles related to pelvic function of the wearer.
